(19) United States
(12) Patent Application Publication
(10) Pub. No.: US 2016/0256481 A1
(43) Pub. Date: Sep. 8, 2016

(54) USE OF WATER SOLUBLE PLATINUM COMPLEX IN PREPARING DRUGS FOR PREVENTION AND TREATMENT OF CANCERS

(71) Applicants: Yiqiang WANG, Tianjin (CN); Yang LIU, Tianjin (CN)

(72) Inventors: Yiqiang WANG, Tianjin (CN); Yang LIU, Tianjin (CN)

(73) Assignee: GUDUI BIOPHARMA TECHNOLOGY INC., Tianjin (CN)

(21) Appl. No.: 14/369,715

(22) PCT Filed: Jun. 22, 2012
(86) PCT No.: PCT/CN2012/077397

§ 371 Date: Nov. 26, 2015

(30) Foreign Application Priority Data
Jun. 24, 2011 (CN) 201110171539.X
Jun. 20, 2012 (CN) 201210206009.9

(51) Int. Cl. A61K 31/7135; A61K 31/519; A61K 31/513

(52) U.S. Cl. CPC A61K 31/7135; A61K 31/513; A61K 31/519

(57) ABSTRACT

Disclosed in the present invention is the use of a water soluble platinum complex in preparing drugs for the prevention and treatment of tumors, said platinum complex being shown as formula (I). Experiments have demonstrated that the present water soluble platinum complex alone or in combination with other chemotherapeutics is able to treat cancer in mammals, and in particular, humans, said cancers including lung cancer, colon cancer, head and neck cancer, prostate cancer, breast cancer, ovarian cancer, cervical cancer, leukemia, lymphoid cancer, skin cancer, pancreatic cancer, liver cancer, bladder cancer, esophageal cancer, gastric cancer, male genital cancer or bone cancer.

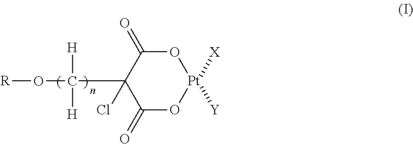

### TECHNICAL FIELD

Certain aspects of the present disclosure provide a method of preventing and treating cancer, especially involving the methods of using water-soluble platinum complexes for prevention and treatment of cancer.

### TECHNICAL BACKGROUND

Cancer is a disease that gene mutation under certain circumstances leads to uncontrolled cell division, thus resulting in continually growth and metastasis of the cell to kill the host. The drugs of prevention and treatment of cancer include alkylating agents, antagonists of cell metabolism, antitumor antibiotics, alkaloids, platinum complexes, as well as asparaginase inhibitors and hormonal therapy and the like. The main purpose of almost all of the antitumor drugs is to effectively prevent the rapid cell division in a short time. So it is often difficult to achieve the aim of high selectivity for killing cancer cells on the aspect of distinguishing tumor cells from normal cells.

Platinum anticancer drugs are a class of representative drugs for prevention and treatment of cancer. They belong to the cell cycle non-specific drugs and the spectrum of antitumor efficacy of them includes solid tumors, malignant epithelial tumor, lymphoma and germ cell tumor and so on. Currently, the representative platinum anticancer drugs widely used in clinical cancer prevention and treatment are cisplatin, carboplatin and oxaliplatin. Cisplatin is the oldest platinum-based anticancer drugs with a long history in clinical use. ((1) Peyrone M. Ann Chemie Pharm (1845), 51, 129 ; (2), Rosenberg, B. &Van Camp, L.; Krigas, T. (1965), “Inhibition of cell division in Escherichia coli by electrolysis products from platinum electrode”, Nature 205 (4972): 698-699), Cisplatin was approved by the FDA as antitumor drug in 1978, its mechanism of action has been well studied and very clearly, which established the foundation for novel structure design and new platinum antitumor drug development, and as a consequence this also actively promote the research on application and development of the platinum-based organometallic compounds in the field of oncology.

The common characteristic drawbacks of the existing platinum drugs is low water solubility which brings lots of disadvantages and difficulties to the stability of its pharmaceutical preparations and clinical application, for example, it is difficult to successfully formulate them as convenient clinical preparations with an appropriate concentration. The water solubility for the clinical platinum antitumor drugs is cisplatin: 1 mg/mL; carboplatin: 17 mg mL; and oxaliplatin: 6 mg/mL, respectively. The low water solubility in combination with the high reactivity toward neucleophilic bases in human body lead to their inevitable and fatal disadvantages, that is the serious adverse side effect including severe renal toxicity and the instability problem of clinical pharmaceutical preparations. ((1), Canetta R, Rozencweig M, Carter S K., Carboplatin: the clinical spectrum to date. , Cancer Treat Rev. (1985), September; 12 Suppl A: 125-36; (2), Knox, R J et al, Mechanism of cytotoxicity of anticancer platinum drugs: evidence that cis-diamminedichloroplatinum(II) and cis-diammine-(1,1-cyclobutanedicarboxylato)platinum(II) differ only in the kinetics of their interaction with DNA., Cancer Res. (1986), April; 46: 1972-9; (3) Overbeck, T, et al. “A comparison of the genotoxic effects of carboplatin and cisplatin in Escherichia Coli”. Mutation Research/DNA Repair. (1996), Volume: 362, Issue: 3, April 2, pp. 249-259; (4) Schnurr, B., Gust, Ronald. “Investigations on the decomposition of carboplatin in infusion solutions”. Mikrochimica Acta. (2002), Volume: 140, Issue: 1-2, August, pp. 69-76).

Studies have shown that platinum anticancer drugs alone can be used effectively damage the DNA of the cancer cell; In addition, in order to further enhance the anticancer efficacy or reduce the potential side effects, the platinum anticancer drugs were widely used in combination with other chemotherapy agents in clinic for prevention and treatment of various cancers. For example, it is well-known that the example of cisplatin can be used in conjunction with 5-fluorouracil to enhance the efficacy of anti-cancer chemotherapy [Cancer Chemotherapy and Pharmacology, Vol.32, p 167, 1993], studies revealed that the pharmacological mechanism is cisplatin can reduce the methionine pool in the cells, which leads to increased methionine biosynthesis and the accumulation and increased concentration of folate cofactors. [ANTICANCER RESEARCH 28: 2373-2378 (2008)]. 5-Fluorouracil metabolites, together with leucovorin and thymidine synthase can form stable complex through covalent bonds, which inhibit the thymidine synthase, thereby prevent DNA replication and synthesis. Based on this mechanism, the synergistic therapeutic new method is developed that cisplatin can be used in combination with 5-fluorouracil to prevent and treat various of solid tumors. [symbol] [symbol] Vol. 18, p 403, 1991; [symbol] Vol.27, p 832, 2000; Investigational New Drugs Vol. 18, p 315, 2000].

However, cisplatin, carboplatin, and oxaliplatin as the existing platinum antitumor drugs have the common characteristic drawbacks of serious side effect and very low water solubility. The major causing factor of the severe renal toxicity of these platinum drugs is that the low water-solublity and high reactivity of the complexes that cause long term accumulation and hard to be excreted from the kindey. Therefore, solve the water solubility problem of the platinum complexes is one of the most important challenges in the field of platinum anticancer drug research and development (Galanski, Markus; Keppler, Bernhard K Searching for the Magic Bullet: Anticancer Platinum Drugs Which Can Be Accumulated or Activated in the Tumor Tissue. Anti-Cancer Agents in Medicinal Chemistry, (2007), 7, 55-73).

### SUMMARY OF THE INVENTION

An object of the present disclosure is to provide a method of using water-soluble platinum complexes for prevention and treatment of cancer.

Another object of the present disclosure is to provide a method of using pharmaceutical composition containing water-soluble platinum complexes for prevention and treatment of cancer.

The technical solutions of the present invention are summarized as follows:

Certain aspects of the present disclosure provide the water solubility platinum complexes of formula (I):

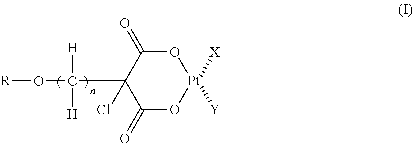

### Wherein:

X and Y are ligands, they are the same or independently chosen from NH₃, a C₁-C₈ aliphatic primary amine, a C₃-C₈ cyclic primary amine, an aromatic amine, an aromatic amine containing at least one C₁-C₄ alkyl-substitution group, or a secondary amine with the formula of R₁-NH-R₂, wherein R₁ and R₂ are the same or different represents a C₁-C₈ aliphatic alkyl group, or together forming a C₄-C₈ cyclic alkyl secondary amine, a nitrogen-con.tainin.g heterocyclic aromatic compound or a nitrogen-con.tainin.g heterocyclic aromatic compound containing at least one C₁-C₄ alkyl-substitution group, a sulfur-containing heterocyclic aromatic compound or a sulfur-containing heterocyclic non-aromatic compound, or X and Y together as of the formula (VIII):

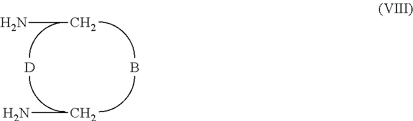

wherein, D is a C₀ or C₁ alkenyl group; B is a C₂-C₈ alkenyl group;

The best example of ligands X and Y includes, but is not limited to: X and Y are each NH₃, isopropylamine, cyclopropylamine, cyclobutylamine, cyclopentylamine, cyclohexylamine; or one of X and Y is NH₃, the other is chosen from isopropylamine, cyclopropylamine, cyclobutylamine, cyclopentylamine, cyclohexylamine, 2-methyl-pyridine; or X and Y together form diamine compound with the formula of H₂N-Z-NH₂, chosen from such as: 1,2-ethylenediamine, 1,3-propanediamine, 2-methyl-tetramethylene diamine, 1,2-cyclohexanediamine, 1,2-cycloheptanediamine, 1,2-cyclooctanediamine, 1-amino-2-(aminomethyl) cyclohexane, 1,1-(diaminomethyl) cyclohexane, 5,5-(diaminomethyl)-1,3-dioxane, 2-(aminomethyl)-pyrrolidine, and 2-(aminomethyl) pyridine. When the above ligand compounds contain chiral centers, they can be any one of optical isomers or racemic mixtures;

Preferably, X and Y are together chosen from trans-(1R,2R)-cyclohexanediamine, trans-(1S,2S)-cyclohexanediamine, cis-(1R,2S)-cyclohexanediamine, cis-(1S,2R)-cyclohexanediamine, racemic trans-1,2-cyclohexanediamine or racemic cis-1,2-cyclohexanediamine.

n is an integer from 1 to 6; Preferably, n is chosen from 1 to 4; the most preferred number is 2 or 3.

R is chosen from the following monosaccharide, the substitution on 1-position of the monosaccharide are α or β or mixture of both.

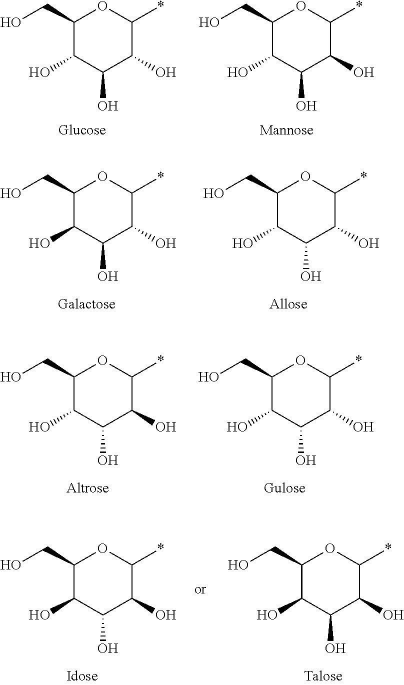

The preferred R is chosen from the following monosaccharide, the substitution on 1-position of the monosaccharide are α or β or mixture of both.

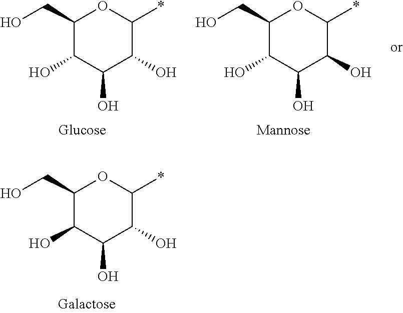 [SMILES: *C1O[C@H](CO)[C@@H](O)[C@H](O)[C@H]1O ; *C1O[C@H](CO)[C@@H](O)[C@H](O)[C@@H]1O ; *C1O[C@H](CO)[C@H](O)[C@H](O)[C@H]1O]

Certain aspects of the present disclosure provide the pharmaceutical composition comprising the water-soluble platinum complex (formula (I)) and at least one other chemotherapeutic agents chosen from the group consisting of Cisplatin, Transplatin, trans-diaminetetrachloroplatinum (IV), Carboplatin, Oxaliplatin, 5-FU, Deoxifluridine, Tegafur, Gemcitabine, Capecitabine, Clofarabine, Temozolomide, Lonafarnib, Erlotinib, Sorafenib, Sunitinib, imatinib, Erlotinib, Bortezomib, Gimatecan, Vinblastine, Vinorelbine, Folinic Acid, Doxorubicin, Paclitaxel, Docetaxel and their diravertives, Tamoxifen, Raloxifene, Spiramycin, Irinotecan. The water soluble platinum complexes as of the formula (I):

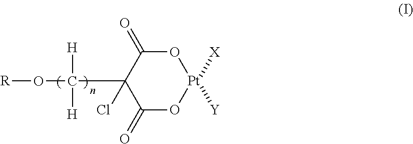

### Wherein:

X and Y are ligands, they are the same or independently chosen from NH₃, a C₁-C₈ aliphatic primary amine, a C₃-C₈ cyclic primary amine, an aromatic amine, an aromatic amine containing at least one C₁-C₄ alkyl-substitution group, or a secondary amine with the formula of R₁-NH-R₂, wherein R₁ and R₂ are the same or different represents a C₁-C₈ aliphatic alkyl group, or together forming a C₄-C₈ cyclic alkyl secondary amine, a nitrogen-containing heterocyclic aromatic compound or a nitrogen-containing heterocyclic aromatic compound containing at least one C₁-C₄ alkyl-substitution group, a sulfur-containing heterocyclic aromatic compound or a sulfur-containing heterocyclic non-aromatic compound, or X an Y together as of the formula (VIII):

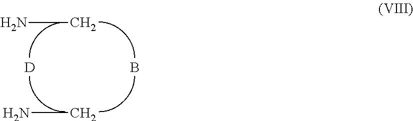

wherein, D is a C₀ or C₁ alkenyl group; B is a C₂-C₈ alkenyl group;

Preferably, X and Y are together chosen from trans-(1R,2R)-cyclohexanediamine, trans-(1S,2S)-cyclohexanediamine, cis-(1R,2S)-cyclohexanediamine, cis-(1S,2R)-cyclohexanediamine, racemic trans-1,2-cyclohexanediamine or racemic cis-1,2-cyclohexanediamine.

n is an integer from 1 to 6; Preferably, n is chosen from 1 to 4; the most preferred number is 2 or 3.

R is chosen from the following monosaccharide, the substitution on 1-position of the monosaccharide are α or β or mixture of both.

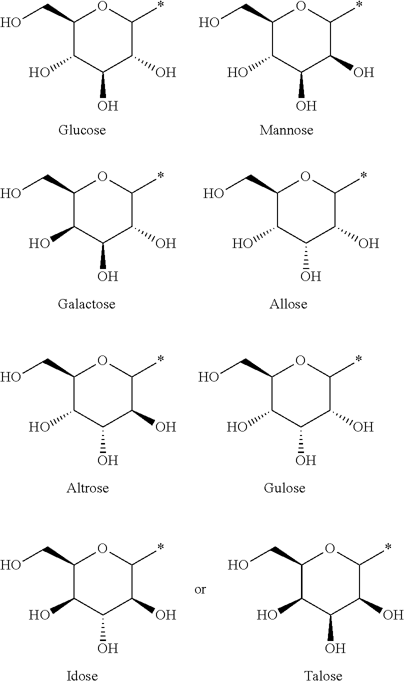

The preferred R is chosen from the following monosaccharide, the substitution on 1-position of the monosaccharide are α or β or mixture of both.

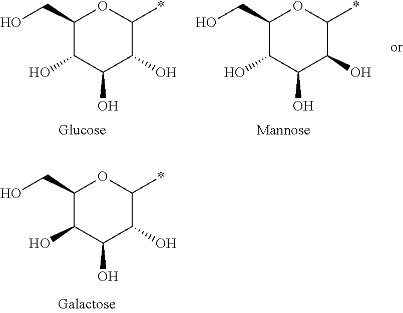 [SMILES: *C1O[C@H](CO)[C@@H](O)[C@H](O)[C@H]1O ; *C1O[C@H](CO)[C@@H](O)[C@H](O)[C@@H]1O ; *C1O[C@H](CO)[C@H](O)[C@H](O)[C@H]1O]

Preferably, the method of using pharmaceutical composition that containing the water-soluble platinum complex to prevents and treats cancer, the pharmaceutical composition is composed of the invented water-soluble platinum complex and 5-fluorouracil, or the water-soluble platinum complex and leucovorin, or the water-soluble platinum complex, 5-fluorouracil and leucovorin.

Certain aspects of the present disclosure provide a method for treating or preventing human lung cancer, human colon cancer, human head and neck cancer, human prostate cancer, human breast cancer, human ovarian cancer, human cervical cancer, leukemia, human lymphoid cancer, human skin cancer, human pancreatic cancer, human liver cancer, human bladder cancer, human esophageal cancer, human gastric cancer, male genital cancer or human bone cancer.

The most preferred is human colorectal cancer.

Certain aspects of the present disclosure provide the advantages of the present invention:

The experiment has demonstrated that the water-soluble platinum complexes of the current invention can prevent and treat cancer in the mammal, for instance, lung cancer, colorectal cancer, head and neck cancer, prostate cancer, breast cancer, ovarian cancer, cervical cancer, leukemia, lymphoid cancer, skin cancer, pancreatic cancer, liver cancer, bladder cancer, esophageal cancer, gastric cancer, male genital cancer, bone cancer and the like. Especially it can prevent and treat human lung cancer, human colon cancer, human head and neck cancer, human prostate cancer, human breast cancer, human ovarian cancer, human cervical cancer, leukemia, human lymphoma, human skin cancer, human pancreatic cancer, human liver cancer, human bladder cancer, human esophageal cancer, human gastric cancer, male genital cancer or human bone cancer.

Due to the synergistic effect can be produced by the combination of the invented water-soluble platinum complex with other chemotherapeutic agents, the pharmaceutical composition comprising the water-soluble platinum complex and further one or more other chemotherapeutic agents exhibit more stronger anticancer efficacy on mammalian lung cancer, colorectal cancer, head and neck cancer, prostate cancer, breast cancer, ovarian cancer, cervical cancer, leukemia, lymphoid cancer, skin cancer, pancreatic cancer, liver cancer, bladder cancer, esophageal cancer, gastric cancer, male genital cancer and bone cancer. Especially it has stronger anticancer efficacy on human lung cancer, human colon cancer, human head and neck cancer, human prostate cancer, human breast cancer, human ovarian cancer, human cervical cancer, leukemia, human lymphoma, human skin cancer, human pancreatic cancer, human liver cancer, human bladder cancer, human esophageal cancer, human gastric cancer, male genital cancer or human bone cancer.

### BRIEF DESCRIPION OF THE DRAWINGS

FIG. 1 shows antitumor efficacy-1 of complex 3;

FIG. 2 shows antitumor efficacy-2 of complex 3;

FIG. 3 shows antitumor efficacy-1 of complex 6;

FIG. 4 shows antitumor efficacy-2 of complex 6;

FIG. 5 shows antitumor efficacy-1 of complex 9;

FIG. 6 shows antitumor efficacy-2 of complex 9;

FIG. 7 shows antitumor efficacy of complex 9, complex 24 and complex 29 in animal tumor-bearing model.

### DETAILED DESCRIPTION

The embodiment described herein will enable the skilled artisan to understand the present invention better instead of limiting the scope of the present disclosure in any way.

Certain aspects of the present disclosure provide the water solubile platinum complexes as of the formula (I):

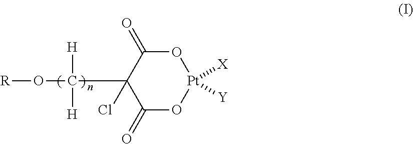

When R in the formula (I) is independently chosen from D-glucose, D-galactose or D-mannose, n and X, Y are shown in table 1

TABLE 1

| n | X | Y |
|---|---|---|
| 1-6 | NH₃ | NH₃ |
| 1-6 | isopropylamine | isopropylamine |
| 1-6 | cyclopropylamine | cyclopropylamine |
| 1-6 | cyclobutylamine | cyclobutylamine |
| 1-6 | cyclopentylamine | cyclopentylamine |
| 1-6 | cyclohexylamine | cyclohexylamine |
| 1-6 | NH₃ | cyclobutylamine |
| 1-6 | NH₃ | cyclopentylamine |
| 1-6 | NH₃ | cyclohexylamine |
| 1-6 | NH₃ | 2-methylpyridine |

| | |
|---|---|
| 1-6 | 1,2-ethylenediamine |
| 1-6 | 1,3-diaminopropane |
| 1-6 | 1,2-cyclobutanediamine |
| 1-6 | 1,2-cyclopentyldiamine |
| 1-6 | 1,2-cycloheptanediamine |
| 1-6 | 1,1-diaminomethylcyclohexane |
| 1-6 | 1,2-diaminomethylcyclobutane |
| 1-6 | 2-aminomethylpyridine |
| 1-6 | 1,2-diaminocyclohexane |

In table 1, the ligand X and Y together form 1,2-cyclohexanediamine, chosen from any one of trans-(1R,2R)-cyclohexanediamine, trans-(1S,2S)-cyclohexanediamine, cis-(1R,2S)-cyclohexanediamine, cis-(1S,2R)-cyclohexanediamine, racemic trans-1,2-cyclohexanediamine, racemic cis-1,2-cyclohexanediamine.

The experiment has shown that the skilled artisan in the domain can prepare all of the complexes in table 1 according to the following disclosed methods.

The water soluble platinum complexes of the formula (I) provided in the present invention can be produced by the following method, the reacting formula as shown below:

Method A:

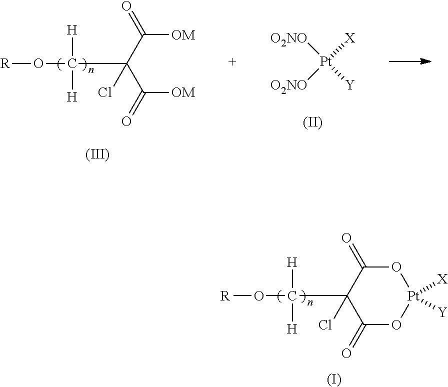

Method B:

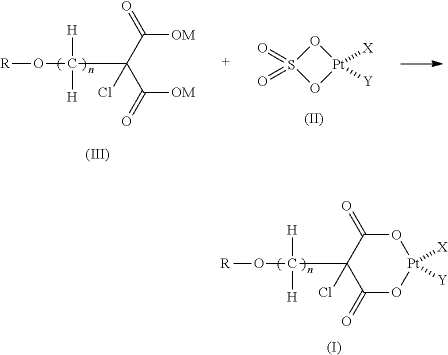

In method A, when M in the formula (III) represents a hydrogen atom, the aqueous solution of the reaction can be adjusted to maintain the PH at 7-9 to complete the preparation of platinum complexes of formula (I). An appropriate inorganic base can be chose to adjust and maintain the pH such as sodium hydroxide, potassium hydroxide, sodium carbonate, sodium bicarbonate, potassium carbonate, lithium hydroxide and cesium hydroxide; when M is represents a metal atom, such as a sodium atom, a potassium atom, M together is a barium atom or a cesium atom, the reaction can be carried out directly in an aqueous solution, if necessary, a small amount of an aqueous solution of the above mentioned inorganic base can be used to maintain the pH of the reaction at 7-9 to finish the preparation of the platinum complexes of the formula (I).

In method B, when M represents a hydrogen atom, the condensation reaction with the platinum sulfate complex as of the formula (II) can be completed in aqueous solution to prepare the desired platinum complexes of formula (I) by using equivalent amount of barium hydroxide as the inorganic base.

Preparation of complexes in the present invention by method B also can be completed by the barium salt (M together represents barium) which prepared in advance, react with the platinum sulfate complexe shown as the formula (II) in an aqueous solution.

The most preferred solvent is deionized water, the reaction carry out at room temperature or heats to 60-90° C. if necessary.

The compounds shown as of the formula (II) in method A and B can be prepared by the reaction of the corresponding complexes of X, Y coordinated cis-platinum dichloride, with silver nitrate or silver sulfate, for example: the cis-dichloro-(1,2-diaminecyclohexane) platinum complex reacts with two equivalents of silver nitrate or one equivalent of silver sulfate to prepare the compounds. The reaction is preferably carried out in an aqueous solution at room temperature, and the most preferred solvent is deionized water.

The compound (II) obtained in above methods then can be used to reacts with the compound (III) which prepared in advance in distilled or deionized water.

0.5-4 equivalents of compound (II) were needed for each equivalent of the compound (III), the preferred amount is 1 to 2 equivalents. The reaction was completed at pH 7-9, which can be maintained by using a suitable base. The best is an inorganic base, such as sodium hydroxide, potassium hydroxide, barium hydroxide, sodium carbonate, potassium carbonate, sodium bicarbonate. The aqueous solution of these bases with approximate equivalent concentration (1 N) was preferably used. The reaction can be carried out within a relatively wide temperature range, for example, 0-100° C., and preferably from room temperature to 90° C., and at the same time with stirring as well. The reaction time varies considerably according to the different target compounds. Depending on the nature of the reactants, the reaction time need generally 1 hour to 30 days, and more often is 10 hours to 15 days.

Many methods can be used to purify the product of formula (I) obtained in the above reaction. For example, the completed reaction mixture can be first filtrated to remove the precipitate that may be generated, and then concentrated by distillation under reduced pressure, and then an organic solvent was added to precipitate out the desired platinum complex of formula (I). An organic solvent which miscible with water is usually selected, such as an alcohol (e.g., methanol, ethanol, propanol, butanol, isopropanol, etc.), or an ether that has a certain miscibility with water (e.g. diethyl ether, methyl tert-butyl ether, THF, ethylene glycol diethyl ether, ethylene glycol dimethyl ether, etc.). Finally the obtained precipitate was collected, for example by filtration, and then the complexes of formula (I) can be achieved. The product obtained above can also be purified and refined by chromatography, etc. for example, by ion exchange resins, or by preparative liquid chromatography. Methanol and water are usually used as mobile phase during separation and purification by liquid chromatography.

In case of glucose, the compound (III) of the present invention can be prepared by each of the following methods: C, D or method E, F.

Method C:

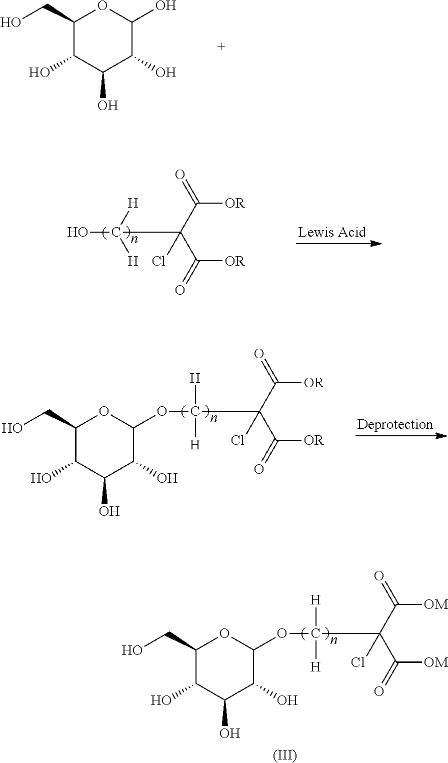

Method D:

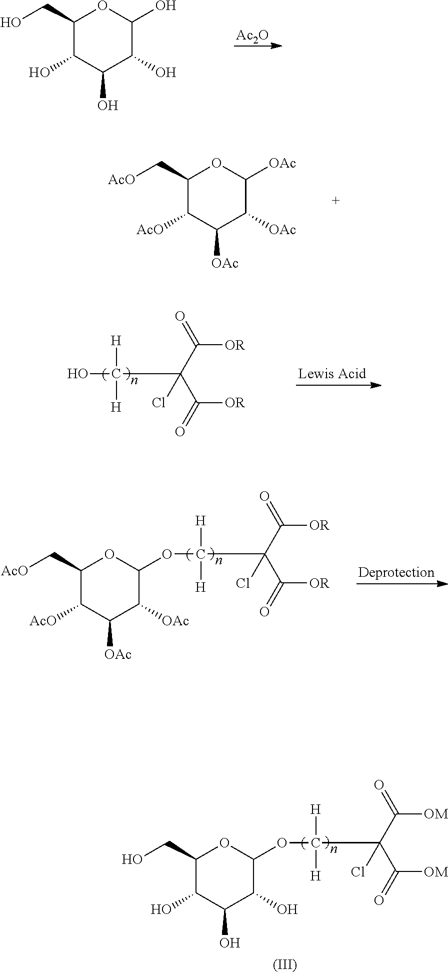

Method E

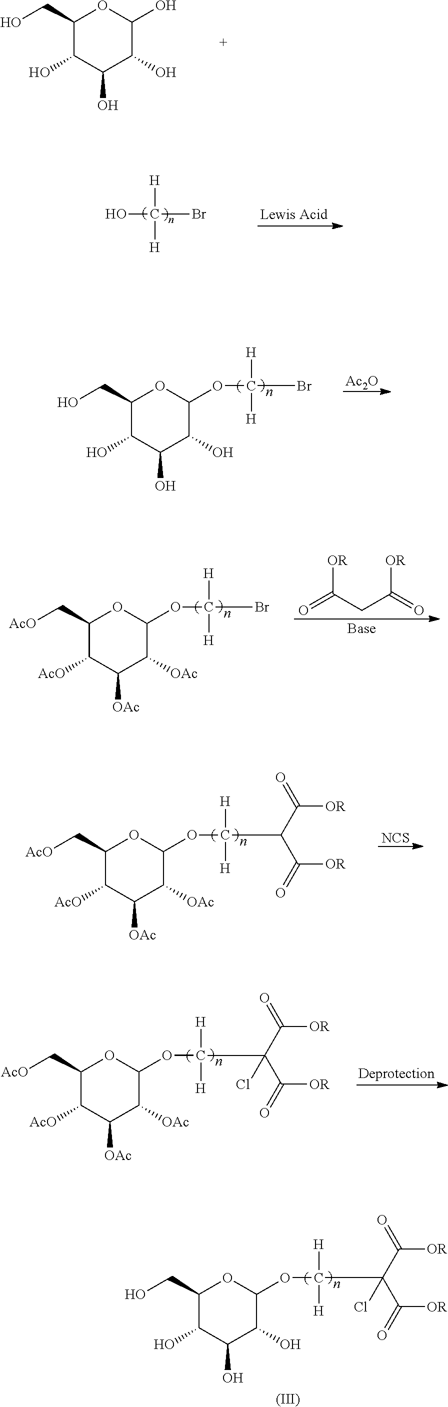

Method F

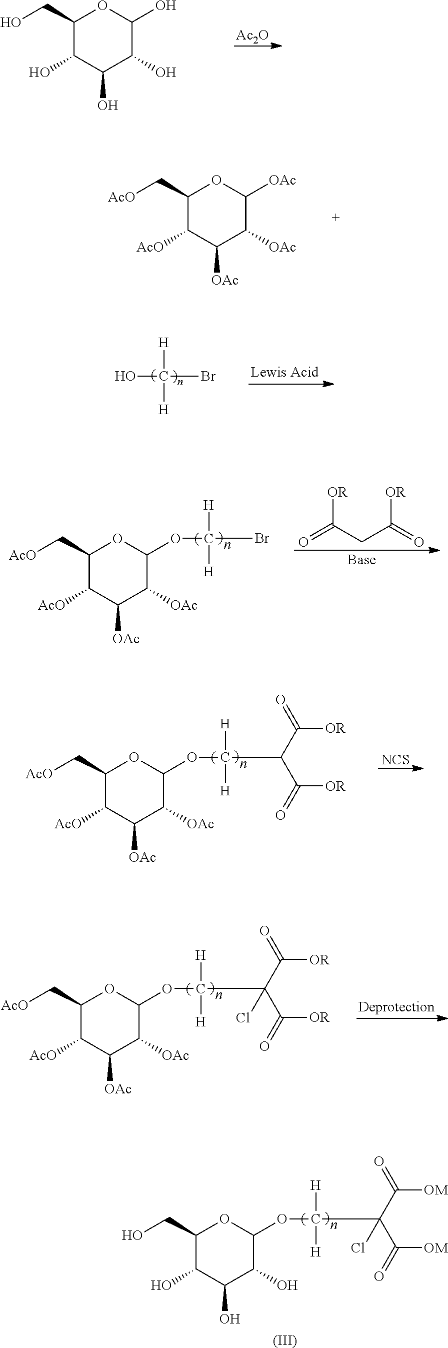

In case of glucose, in method C, 2-chloro substituted malonate derivatives, can be prepared by reacting a halogenated alkyl alcohol with 2-chloromalonate derivatives such as dimethyl chloromalonate, diethyl chloromalonate, dibenzyl chloromalonate and 2-chloromalonic acid cyclic isopropylidene ester and the like according to the general methods known in the literature (e.g. Journal of the American Chemical Society, 131(8), 2786-2787: 2009). Then condensation reaction of the resulting 2-chloro-2-hydroxyalkyl malonate derivatives with D-glucose in the presence of a Lewis acid can produce the corresponding glucoside compounds. 0.1-50 equivalents of 2-chloromalonate derivatives in respect to glucose was used in the condensation reaction, or on the contrary, 0.1-50 equivalents of glucose in respect to 2-chloromalonate compunds was used. The Lewis acid may be chosen from BF₃, SnCl₄, FeCl₃, AlCl₃, hydrochloric acid, p-toluenesulfonic acid, camphorsulfonic acid, etc. The amount of Lewis acid can be 0.1-10 equivalents in respect to glucose. The solvent can be selected from THF, dichloromethane, toluene, ethylene glycol dimethyl ether, ethylene glycol diethyl ether, etc. Any one of the two reactants can also be chose as the solvent. The reaction temperature can be from 0 to 100° C., generally at 60-80° C. The reaction time differs depending on the reactants, generally ranging from 1 hour to 7 days. The resulting products can be refined by a series of purification methods, generally by silica gel column chromatography or by liquid chromatography. The obtained product, after removal of the protecting group of malonic acid, can give the desired compounds shown as the formula (III). The method of deprotection differs depending on the protecting group, for example, benzyl group can be removed by hydrogenation, and diethyl group and isopropylidene group can be deprotected by using an inorganic base with methanol-water, or THF-water as solvent, the ratio of organic solvent to water is generally 1:1-4:1. The inorganic bases can be chose from sodium hydroxide, potassium hydroxide, barium hydroxide and lithium hydroxide, etc. The reaction temperature for the deprotection reaction is usually from room temperature to 60° C., the reaction time generally ranges from 1 to 24 hours. The compound after deprotection can be purified by silica gel column chromatography or ion exchange resin, or by liquid chromatography. If the reaction solvent can be removed directly by distillation, the resulting product will be the corresponding metal carboxylate salt of formula (III).

As shown in Method D, D-glucose can also be firstly converted into the corresponding acetylated glucose, and then react with the 2-chloromalonate derivatives. D-glucose can be acetylated in accordance with the method reported in the literature, for example, the acetylation can be completed in pyridine with acetic anhydride as the acetylating agent at room temperature or at 60° C. for 1-24 hours. Other steps and conditions except the acetylation in Method D, are the same as described in Method C.

In method E and F, halohydrin are firstly coupled with glucose or acetylated glucose in the presence of a Lewis acid, and then the obtained glucoside react with malonate followed by the chlorination, and finally produce the compound (III). Chloro substitution at 2-position of malonate can be accomplished by using the NCS as a representative chlorinating reagent. The reaction is completed in DMF, THF or diethyl ether by treating malonate with one equivalent or excess amount of the base and then the chlorinating reagent. The base may be chosen from sodium hydride, potassium carbonate, sodium carbonate, cesium carbonate, sodium bicarbonate, etc. The equivalent of chlorinating reagent is 1-3 times of the malonate, the reaction temperature is generally from 0° C. to 60° C., preferably at room temperature with stirring. Except the chlorination reaction, all other reaction conditions involved in acetylation of glucose, glycosidation reaction in the presence of Lewis acid, base mediated alkylation reaction at 2-position of the malonate and the final deprotection reaction, are the same as described in method C and D.

### EXAMPLE 1

Anti-proliferation effects of the invented water-soluble platinum complexes.

The following experiments have been done to testify the proliferation inhibition effect of the water-soluble platinum complexes on different types of human tumor cells.

(1) Test Methods:

Cell Culture Medium:

Containing 10% bovine fetal serum, 1 mM of sodium pyruvate, 2 mM of L-glutamine, 50 U/mL of penicillin, 50 μg/mL of streptomycin

The main experimental apparatus:

MCO-15A Carbon Dioxide Incubator (SANYO, Japan), Inverted Phase Contrast Microscope (Olympus, Japan), Automatic Microplate Reader (U.S. BioTEK ELX808), Low Temperature Refrigerator (MDF-V5410, Japan), Clean Bench (Suzhou Medical Apparatus Factory), Micropipettes (GILSON, France), Automatical Pure Water Distillatory (1810B, Shanghai).

Reagents:

MTS: CellTiter96 Aqueous MTS Reagent Powder, Promega

PMS: Phenazine methosulfate (PMS), Sigma-Aldrich

DPBS: Sigma-Aldrich

Tumor Cells:

Human tumor cells: du145-human prostate cancer; MCF-7-human breast cancer; SK0V3-human ovarian carcinoma, HT-29-human colon cancer; A549-human non-small cell lung cancer (adenocarcinoma), H460-human non-small cell lung cancer (large cell carcinoma), and animal tumor cells: L 1210-mice leukemia cells used in the following activity test experiments were all purchased from Shanghai An Yan Commercial Trade Co., Ltd.

Cytotoxicity Test:

MTS test method was used in cytotoxicity assay. The tumor cells of logarithmic phase were collected, and then the concentration of cell suspension was adjusted, 100 μL of the cell suspension was added to each well, the cells were placed at 1000-10000 cells/well (edge well filled with sterile PBS). Cells were incubated at 37° C. with 5% CO₂ to make cell monolayer overspread the bottom of each well (96-well flat-bottomed microplate). 100 μL of different concentrations of the test compounds was added to each well. Each condition was measured in five replicates. The microplate was incubated at 37° C. with 5% CO₂ for 96 h and checked with inverted microscope. MTS working reagent: To 2 mL of MTS (2 mg/mL, prepared by DPBS) was added 100 μL of PMS (1 mg/mL, prepared by DPBS). The cell culture medium was discarded after centrifugation, the cell culture plate was carefully washed 2-3 times with PBS. Before detecting the absorbance value (OD), to each sample containing well was added 100 μL of cell culture medium, then 20 μL of MTS working reagent was added. After incubation at 37° C. with 5% CO₂ for 2 h, the OD (optical density) value was detected at 490 nm.

Control group: the conditions are the same as the above without adding the active ingredient of antitumor agents, and the OD value was detected at 490 nm on the end of the experiment.

Cyototoxicity IC₅₀:

The cell inhibiting rate of the drugs to tumor cell growth was calculated according to the following formula:

1) Cell viability (%)=OD of treated group/OD of control group×100%

2) The cell viabilities under the different drug concentrations were determined, and then plotted against drug concentration. The IC₅₀ value is the corresponding concentration in the obtained curve when the cell viability was 50%.

(2) Test Complexes:

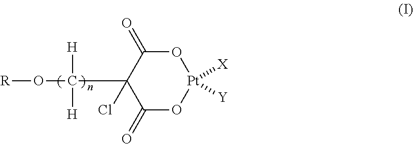

TABLE 2
Complexes of the experiment

| Complexs | Monosaccharide | n | X | Y |
|---|---|---|---|---|
| 1 | Glucose | 1 | Trans-(1R,2R)-cyclohexane | |
| 2 | Glucose | 2 | Trans-(1R,2R)-cyclohexane | |
| 3 | Glucose | 3 | Trans-(1R,2R)-cyclohexane | |
| 4 | Mannose | 1 | Trans-(1R,2R)-cyclohexane | |
| 5 | Mannose | 2 | Trans-(1R,2R)-cyclohexane | |
| 6 | Mannose | 3 | Trans-(1R,2R)-cyclohexane | |
| 7 | Galactose | 1 | Trans-(1R,2R)-cyclohexane | |
| 8 | Galactose | 2 | Trans-(1R,2R)-cyclohexane | |
| 9 | Galactose | 3 | Trans-(1R,2R)-cyclohexane | |
| 10 | Glucose | 1 | NH₃ | NH₃ |
| 11 | Glucose | 2 | NH₃ | NH₃ |
| 12 | Glucose | 3 | NH₃ | NH3 |
| 13 | Mannose | 1 | NH₃ | NH3 |
| 14 | Mannose | 2 | NH₃ | NH3 |
| 15 | Mannose | 3 | NH₃ | NH3 |
| 16 | Galactose | 1 | NH3 | NH3 |
| 17 | Galactose | 2 | NH3 | NH3 |
| 18 | Galactose | 3 | NH3 | NH3 |
| 19 | Glucose | 1 | 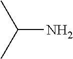 | 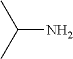 |
| 20 | Glucose | 2 | 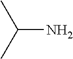 | 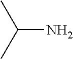 |
| 21 | Glucose | 3 | 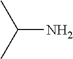 | 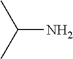 |
| 22 | Mannose | 1 | 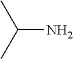 | 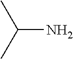 |
| 23 | Mannose | 2 | 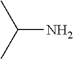 | 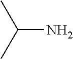 |
| 24 | Mannose | 3 | 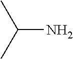 | 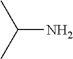 |
| 25 | Galactose | 1 | 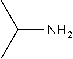 | 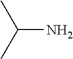 |
| 26 | Galactose | 2 | 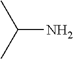 | 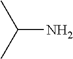 |
| 27 | Galactose | 3 | 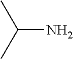 | 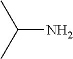 |
| 28 | Glucose | 1 | 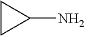 | 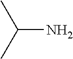 |
| 29 | Glucose | 2 | 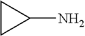 | 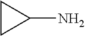 |
| 30 | Glucose | 3 | 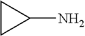 | 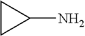 |
| 31 | Mannose | 1 | 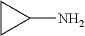 | 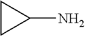 |
| 32 | Mannose | 2 | 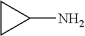 | 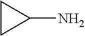 |
| 33 | Mannose | 3 | 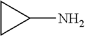 | 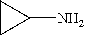 |
| 34 | Galactose | 1 | 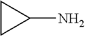 | 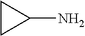 |
| 35 | Galactose | 2 | 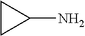 | 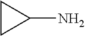 |
| 36 | Galactose | 3 | 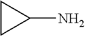 | 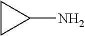 |

(3) Experimental Results:

Symbols representing the names of tumor cells are as follows: du145-human prostate cancer; MCF-7-human breast cancer; SKOV3-human ovarian carcinoma; HT-29-human colon cancer; A549-human non-small cell lung cancer (adenocarcinoma); H460-human non-small cell lung cancer (large cell carcinoma)

TABLE 3
The IC₅₀ value of the complexes in
different human tumor cells (μM)

| Tumor cell | A549 | SKOV3 | MCF7 | HT29 | DU145 | H460 |
|---|---|---|---|---|---|---|
| Complex 2 | 0.821 | 10.000 | 1.999 | 1.876 | 5.186 | 22.010 |
| Complex 5 | 1.100 | 5.037 | 0.525 | 2.046 | 9.909 | 22.130 |
| Complex 8 | 10.260 | 22.100 | 1.065 | 1.325 | 4.943 | 10.070 |

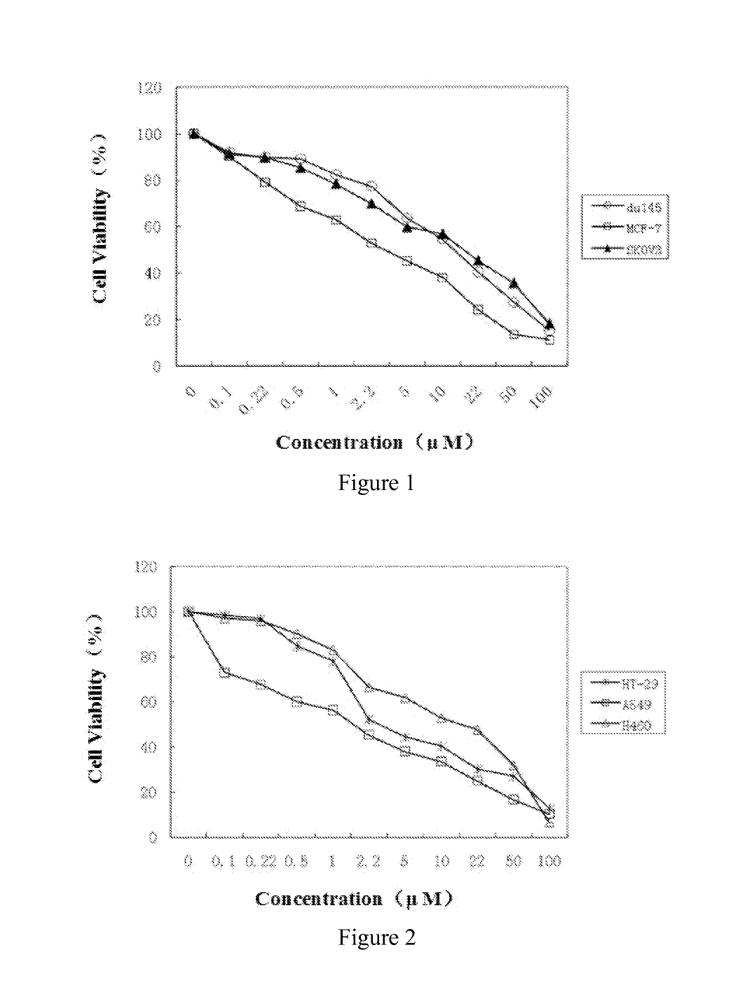

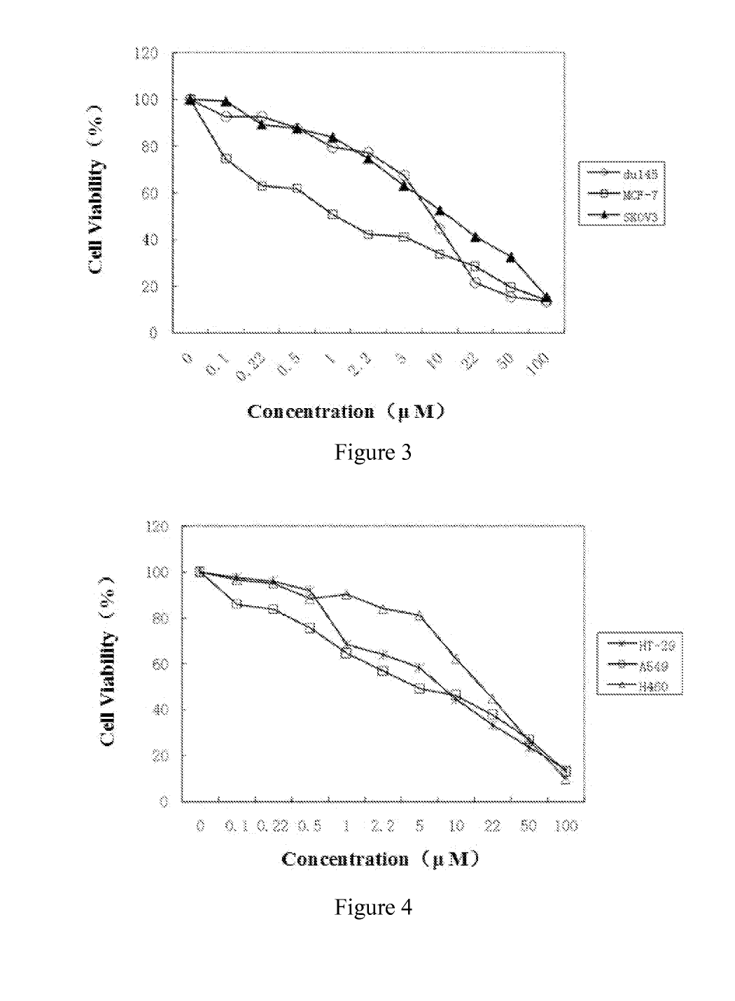

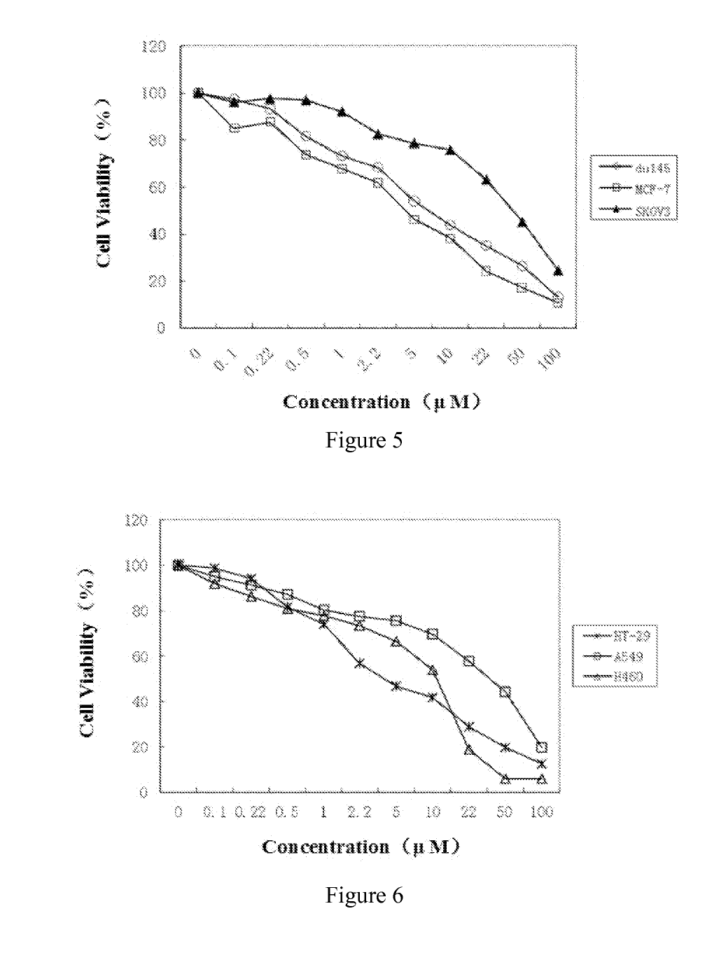

The anti-tumor effect of the Complex 3 is shown in FIG. 1 and FIG. 2; the anti-tumor effect of the Complex 6 is shown in FIG. 3 and FIG. 4; the anti-tumor effect of the Complex 9 is shown in FIG. 5 and FIG. 6. In order to show the efficacy trend of the complexes more clearly, standard error bar in all curves in the graph is omitted.

### EXAMPLE 2

Anti-proliferation effects of the invented water-soluble platinum complexes in combination with other chemotherapy drugs (active ingredient). The following experiment has studied the synergistic effects of the invented water-soluble platinum complexes in combination with other chemotherapy drugs (active ingredient).

(1) Test Method:

Cell Culture Medium:

Containing 10% bovine fetal serum, 1 mM of sodium pyruvate, 2 mM of L-glutamine, 50U/mL of penicillin, 50 mL of streptomycin. The main experimental apparatus:

MCO-15A Carbon Dioxide Incubator (SANYO, Japan), Inverted Phase Contrast Microscope (Olympus, Japan), Automatic Microplate Reader (U.S. BioTEK ELX808), Low Temperature Refrigerator (MDF-V5410, Japan), Clean Bench (Suzhou Medical Apparatus Factory), Micropipettes (GILSON, France), Automatical Pure Water Distillatory (1810B, Shanghai).

Reagents:

MTS: CellTiter96 Aqueous MTS Reagent Powder, Promega

PMS: Phenazine methosulfate (PMS), Sigma-Aldrich

DPBS: Sigma-Aldrich

Tumor Cells:

Human tumor cells: du145-human prostate cancer; MCF-7-human breast cancer; SKOV3-human ovarian carcinoma, HT-29-human colon cancer; A549-human non-small cell lung cancer (adenocarcinoma), H460-human non-small cell lung cancer (large cell carcinoma), and animal tumor cells: L 1210-mice leukemia cells used in the following activity test experiments were all purchased from Shanghai An Yan Commercial Trade Co., Ltd.

Synergistic Cytotoxicity Test:

MTS test method was used in cytotoxicity assay. The tumor cells of logarithmic phase were collected, and then the concentration of cell suspension was adjusted, 100 μL of the cell suspension was added to each well, the cells were placed at 1000-10000 cells/well (edge well filled with sterile PBS). Cells were incubated at 37° C. with 5% CO2 to make cell monolayer overspread the bottom of each well (96-well flat-bottomed microplate). 100 μL of certain concentration solution of a mixture of the water-soluble platinum complex with other antitumor agents was added to each well. Each condition was measured in five replicates. The microplate was incubated at 37° C. with 5% CO₂ for 96 h and checked with inverted microscope. MTS working reagent: To 2 mL of MTS (2 mg/mL, prepared by DPBS) was added 100 μL of PMS (1 mg/mL, prepared by DPBS). The cell culture medium was discarded after centrifugation, the cell culture plate was carefully washed 2-3 times with PBS. Before detecting the absorbance value (OD), to each sample containing well was added 100 μL of cell culture medium, then 20 μL of MTS working reagent was added. After incubation at 37° C. with 5% CO₂ for 2 h, the OD (optical density) value was detected at 490 nm.

Each of above experiment was repeated five times, and the cell viability was calculated according to the mean OD values.

Cell viability was calculated using the following formula:

Cell Viability (%)=(treated group OD value/control group OD value)×100

Control group: the conditions are the same as the above without adding the tested drug mixture (100 μL of culture medium was added in stead), and finally the absorbance was measured at 490 nm to obtain the OD value of the tumor cells.

Drug Group-1: only the water-soluble platinum complex was added under the above conditions to obtain cell viability of tumor cells.

Drug Group-2: only the other chemotherapeutic drug (active ingredient) was added under the above conditions to obtain cell viability of tumor cells.

Drug Combination Group: the water-soluble platinum complex and other chemotherapeutic drugs were added simultaneously under the above conditions to obtain cell viability of tumor cell.

(2) Evaluation Method:

The combination effect of the platinum complexes:

When the water-soluble platinum complexes used in combination with other chemotherapy drugs, the inhibition effect on cancer cell proliferation is enhanced or synergistic, and the effect was calculated using the following formula:

Combination Effect (%)={[(A1−X)+(A2−X)]/(A1−A2)|}×100

wherein, A1 is the cell viability of drug group-1, A2 is the cell viability of drug group-2, X is the cell viability of drug combination group,(A1-A2)| is the absolute value of the cell viability difference between the two groups. Calculated according to the above formula, when the results of [Combination Effect (%)]>+100%, that means the effect of inhibition on cell proliferation is enhanced or synergistic

(3) Experimental Result:

TABLE 4
the effect of drug combinations between the
complex 2 and other chemotherapy drugs.

| Other | The dosage | | | | | |
|---|---|---|---|---|---|---|
| chemo- | of other | | | | | |
| therapy | chemotherapy | | | | | |
| drug (active | drug (active | Complex 2 (dosage: 1 μM) | | | | |
| ingredient) | ingredient) | A549 | SLOV3 | MCF7 | HT29 | DU145 |
| Transplatin | 10 μM | ⊚ | ◯ | ⊚ | ⊚ | ◯ |
| Irinotecan | 20 μM | ◯ | ⊚ | ◯ | ◯ | ◯ |
| Gemcitabine | 1 μM | ⊚ | ⊚ | ⊚ | ⊚ | ⊚ |
| Capecitabine | 200 μM | ◯ | ◯ | ⊚ | ⊚ | ◯ |
| 5- | 15 μM | ⊚ | ⊚ | ⊚ | ⊚ | ⊚ |
| Fluorouracil | | | | | | |
| 5-FU + | 15 μM + 5 μM | ⊚ | ⊚ | ◯ | ⊚ | ⊚ |
| Leucovorin | | | | | | |
| Paclitaxel | 1 μM | ⊚ | ⊚ | ⊚ | ⊚ | ◯ |
| Tarceva | 1 μM | ⊚ | ◯ | ◯ | ◯ | ◯ |

*In the table, ⊚ represents the effect of drug combination >300%; ◯ represents: 100% < the effect of drug combination < 300%.

TABLE 5
the effect of drug combination between the
complex 3 and other chemotherapy drugs.

| Other | The dosage | | | | | |
|---|---|---|---|---|---|---|
| chemo- | of other | | | | | |
| therapy | chemotherapy | | | | | |
| drug (active | drug (active | Complex 3 (dosage: 1 μM) | | | | |
| ingredient) | ingredient) | A549 | SlOV3 | MCF7 | HT29 | DU145 |
| Transplatin | 10 μM | ⊚ | ◯ | ⊚ | ⊚ | ⊚ |
| Irinotecan | 20 μM | ⊚ | ⊚ | ◯ | ⊚ | ◯ |
| Gemcitabine | 1 μM | ⊚ | ◯ | ⊚ | ⊚ | ⊚ |
| Capecitabine | 200 μM | ◯ | ◯ | ⊚ | ◯ | ◯ |
| 5- | 15 μM | ⊚ | ⊚ | ⊚ | ⊚ | ⊚ |
| Fluorouracil | | | | | | |
| 5-FU + | 15 μM + 5 μM | ⊚ | ⊚ | ⊚ | ⊚ | ⊚ |
| Leucovorin | | | | | | |
| Paclitaxel | 1 μM | ⊚ | ⊚ | ⊚ | ⊚ | ⊚ |
| Tarceva | 1 μM | ⊚ | ◯ | ◯ | ⊚ | ◯ |

*In the table, ⊚ represents the effect of drug combination >300%; ◯ represents: 100% < the effect of drug combination < 300%.

TABLE 6
the effect of drug combination between the
complex 5 and other chemotherapy drugs.

| Other | The dosage | | | | | |
|---|---|---|---|---|---|---|
| chemo- | of other | | | | | |
| therapy | chemotherapy | | | | | |
| drug (active | drug (active | Complex 5 (dosage: 1 μM) | | | | |
| ingredient) | ingredient) | H460 | SLOV3 | MCF7 | HT29 | DU145 |
| Transplatin | 10 μM | ⊚ | ◯ | ⊚ | ⊚ | ⊚ |
| Irinotecan | 20 μM | ⊚ | ⊚ | ◯ | ⊚ | ◯ |
| Gemcitabine | 1 μM | ⊚ | ◯ | ⊚ | ⊚ | ◯ |
| Capecitabine | 200 μM | ◯ | ◯ | ⊚ | ◯ | ◯ |
| 5- | 15 μM | ⊚ | ⊚ | ⊚ | ⊚ | ⊚ |
| Fluorouracil | | | | | | |
| 5-FU + | 15 μM + 5 μM | ⊚ | ⊚ | ⊚ | ⊚ | ⊚ |
| Leucovorin | | | | | | |
| Paclitaxel | 1 μM | ⊚ | ⊚ | ⊚ | ⊚ | ⊚ |
| Tarceva | 1 μM | ⊚ | ◯ | ◯ | ⊚ | ◯ |

*In the table, ⊚ represents the effect of drug combination >300%; ◯ represents: 100% < the effect of drug combination < 300%.

TABLE 7
the effect of drug combination between the
complex 6 and other chemotherapy drugs.

| Other | The dosage | | | | | |
|---|---|---|---|---|---|---|
| chemo- | of other | | | | | |
| therapy | chemotherapy | | | | | |
| drug (active | drug (active | Complex 6 (dosage: 1 μM) | | | | |
| ingredient) | ingredient) | H460 | SLOV3 | MCF7 | HT29 | DU145 |
| Transplatin | 10 μM | ⊚ | ◯ | ⊚ | ⊚ | ⊚ |
| Irinotecan | 20 μM | ⊚ | ⊚ | ◯ | ⊚ | ⊚ |
| Gemcitabine | 1 μM | ⊚ | ◯ | ⊚ | ⊚ | ◯ |
| Capecitabine | 200 μM | ◯ | ◯ | ⊚ | ◯ | ◯ |
| 5- | 15 μM | ⊚ | ⊚ | ⊚ | ⊚ | ⊚ |
| fluorouracil | | | | | | |
| 5-FU + | 15 μM + 5 μM | ⊚ | ⊚ | ⊚ | ⊚ | ⊚ |
| Leucovorin | | | | | | |
| Paclitaxel | 1 μM | ⊚ | ⊚ | ⊚ | ◯ | ⊚ |
| Tarceva | 1 μM | ⊚ | ◯ | ◯ | ⊚ | ◯ |

*In the table, ⊚ represents the effect of drug combination >300%; ◯ represents: 100% < the effect of drug combination < 300%.

### EXAMPLE 3

In the following test, antitumor efficacy studies were performed using 8-9 weeks old female CDF1 mice, the average weight of the animal is 20-25 grams. L1210 tumor cells (about 105 cells per mouse) were inoculated intraperitoneally. The water-soluble platinum complexes were used to treat the tumor-bearing animals and the efficacy was compared compared with the clinical platinum antitumor drugs. For the water-soluble platinum complexes of the present invention and carboplatin, 5 wt % mannitol-water solution was used for preparing the corresponding injection, but for cisplatin, 5 wt % mannitol-saline solution was used. Drugs were administered intraperitoneally on day 1 and day 4 after tumor cell transplantation. The number of experimental animals in each group was 6.

The experimental animals were purchased from Vital River Laboratory Animal Technology Co. Ltd., Tumor cells L1210-leukemia cells were purchased from Shanghai An Yan Commercial Trade Co. Ltd.

The increase in life span (ILS) is calculated as follows:

ILS%=[(St/Su)−1]×100%

Wherein, St=the weighted median survival time of treated animals; Su=the weighted median survival time of untreated animals

The results are shown in Table 8:

TABLE 8

| | | | | Survived |
|---|---|---|---|---|
| | Dosage | Weight | | Animals on |
| Compound | (mg/Kg) | Changes (g)* | ILS (%) | Day 42 |
| Untreated Control | / | +1.8 | / | 0/6 |
| Group | | | | |
| Complex-2 | 50 | +1.1 | >425 | 6/6 |
| | 100 | +0 | >425 | 6/6 |
| | 200 | −0.4 | >425 | 6/6 |
| Complex-6 | 50 | +1.2 | >365 | 6/6 |
| | 100 | −0.2 | >365 | 6/6 |
| | 200 | −0.5 | >365 | 6/6 |
| Carboplatin | 24 | +0.6 | 8 | 0/6 |
| | 40 | −0.4 | 22 | 0/6 |
| | 80 | −1.2 | 50 | 1/6 |
| Cisplatin | 1.25 | +1.1 | 20 | 0/6 |
| | 2.5 | −0.6 | 125 | 1/6 |
| | 5.0 | −2.2 | 265 | 3/6 |

*weight changes from the first day to the seventh day

### EXAMPLE 4

Anti-tumor effect of the water-soluble platinum complexes of the present invention on animal xenograft models

(1) Test Methods:

Anti-tumor effect studies were performed using 5-6 weeks old Nu/nu male nude mice which were purchased from Beijing Vital River Laboratory Animal Technology Co. Ltd. Experimental animals were kept under the SPF level environment in the IVC systems. All animals had a free access to the food and water, the room temperature was 20 to 25° C., the humidity was 40% to 70%, and the alternation of day and night was 12 h/12 h.

Colorectal cancer DLD-1 cells were collected and subcutaneously injected into the armpit of each nude mouse, and then the model of tumor bearing mice was established. When the tumor volume grew to 150˜300 cm³, according to the tumor volume and weight, the mice were equally divided into 5 groups (saline group, Complex-9 group, Complex-24 group, Complex-29 group, oxaliplatin group, 10 animals in each group). Experimental compounds were injected intraperitoneally once a week, and the volume of administration is 10 mL/kg body weight. After four weeks of the drug treatment, the mice were continually fed with a normal diet, the tumor growth and the anti-tumor efficacy of the tested compounds were dynamically observed by measuring tumor volume and size on alternate days. Experimental observation was continued for 61 days after grouping.

The calculation formula of tumor volume: V=1/2×a×b². Wherein, a and b are the tumor length and width, the tumor volume was calculated based on the measurements. Percent tumor volume increase (%)=(Vₜ−V₀)/V₀×100. V₀ is the tumor volume before administration (that is d₀); Vₜ is the tumor volume after administration.

(2) Administration Dosage:

According to the pre-measured maximum tolerated dose (MTD) of the drugs on the same nude mice, 70% of the MTD was used as the administration dosage. Wherein clinical oxaliplatin was 7.5 mg/kg body weight, the platinum complex of Complex-9 was 45 mg/kg body weight, the platinum complex of Complex-24 was 28 mg/kg body weight, and the platinum complex of Complex-29 was 20 mg/kg body weight. Drugs were dissolved in sterile distilled water using ultrasound before injection.

(3) Experimental Results:

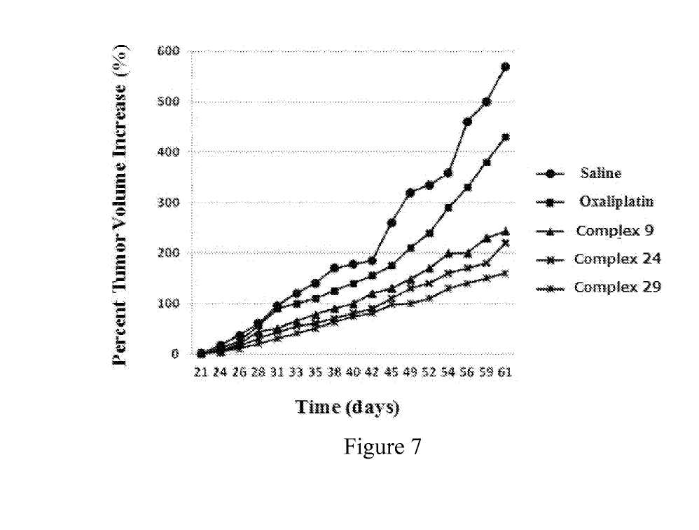

the experimental results show that the water-soluble platinum complexes of the present invention have a significant advantage over clinical drug oxaliplatin on tumor inhibition effect, especially in aspect of long term suppression effect on tumor growth after drug treatment being stopped. This result implicates that, in spite of the extremely high water solubility (theoretically can not cross over the cell membrane), the invented platinum complexes have selected accumulation in tumor cells and tissues and therefore, exhibited improved tumor targeting effect (See FIG. 7). In order to clearly show the efficacy trend of the complexes, standard error bar in all curves in the graph is omitted.

The pharmaceutical composition comprising any one of the water-soluble platinum complexes shown in the formula (1) can be used in combination with one or more chemotherapeutic drugs, antiemetics, antidotes, anti-ulcer drugs and the like. The chemotherapeutic drugs can be chosen from Cisplatin, Transplatin, trans-diaminetetrachloroplatinum (IV), Carboplatin, Oxaliplatin, 5-FU, Deoxifluridine, Tegafur, Gemcitabine, Capecitabine, Clofarabine, Temozolomide, Lonafamib, Erlotinib, Sorafenib, Sunitinib, Imatinib, Erlotinib, Bortezomib, Gimatecan, Vinblastine, Vinorelbine, Folinic Acid, Doxorubicin, Paclitaxel, Docetaxel and their diravertives, Tamoxifen, Raloxifene, Spiramycin, Irinotecan, and so on.

The preventive effect on cancer of the water-soluble platinum complexes means that the water-soluble platinum complexes alone or used in conjunction with other chemotherapeutic drugs can kill or remove or suppress cancer cells therefore, prevent the tumor metastasis and endangering the health and life of the host.

### Method of the Therapy

The water-soluble platinum complexes shown in the formula (I) can nbe used to prepare a pharmaceutical composition for prevention and treatment of cancer. The pharmaceutical compositions comprise a therapeutically effective amount of water-soluble platinum complexes together with a pharmaceutically acceptable carrier or diluter. The pharmaceutical acceptable carrier or diluter include such as glucose, dextrin, fructose, maltose, lactose, gelatin, sucrose, hydroxyl cellulose, hydroxypropylmethyl cellulose, silicon dioxide, stearic acid, sodium starch glycollate, water, ethanol, sodium chloride etc. and they may be selected depending on the needs of the dosage form. In addition, according to the need of the pharmaceutical preparation, the pharmaceutical excipients also include a small amount of pH buffering agents, stabilizer and the like.

In the methods of cancer prevention and treatment using the water-soluble platinum complex shown in the formula (I), the water-soluble platinum complexes can be used in injectable form depending on the needs of treatment. The injection is sterile and isotonic with blood. When the flyophilized powder of the water-soluble platinum complexes are used, the diluter can be chosen from 5% of dextrose injection solution, 0.9% of sodium chloride injection solution, 5% of glucose saline injection solution, 5% of glucose Ringer's Injection solution and so on to make the lyophilized powder component diluted into clinically allowed capacity to implement the treatment. When it is necessary, buffers and alleviative agents can be added in addition to the above mentioned pharmaceutical acceptable diluents.

### Dosage

When the water-soluble platinum complex shown in the formula (I) as an active ingredient is used alone to prevent and treat cancer, the administration dosage is adjusted according to the patient's age, weight, sex and status of the patients. Generally, for an adult patient, the range of the administration is between 10 mg and 1000 mg one time, once per week or several injections per month.

When water-soluble platinum complex shown in the formula (I) was used in combination with other chemotherapy drugs, the administration dosage of the other chemotherapeutic drugs was generally according to the dosage in their clinical specification.

### The Physical and Chemical Parameters of the Complexes

The main experimental apparatus:

NMR spectrometer: BRUKER AVANCE III, 400 MHz; Liquid Chromatography for Analysis: Beijing Tong Heng Innovation LC3000 high performance liquid chromatography, with SPD-10ATvp dual wavelength UV detector, 7725i manual injector, CLASS-VP chromatography workstation; Analytical HPLC Column: DaisoGel, C₁₈, 4.6×250 cm, 5 μm KNAUER Germany; Semi-preparative Liquid Chromatography: Beijing Tong Heng Innovation LC3000 semi-preparative liquid chromatography, SPI001; Semi-preparative Column: DaisoGel 250×20 mm ID, C18, 10 μm; Mass Spectrometer: Agilent 6310 Ion Trap LC/MS; Lyophilizer: FD-lc-50 lyophilizer (Beijing Boyikang Laboratory Instruments Co., Ltd).

Complex 1:

¹H NMR (400 MHz, D₂O), ppm: 4.87 (d, J=3.6 Hz, 0.8H); 4.43 (d, J=7.2 Hz, 0.2H); 3.00-4.50 (m, 8H); 2.20-2.45 (m, 2H); 1.96 (d, J=12 Hz, 2H); 1.49 (d, J=8 Hz, 2H); 1.12-1.30 (s, 2H); 0.95-1.10 (m, 2H); MS: m/z: 638.16 [M+H]⁺.

Complex 2:

¹H NMR (400 MHz, D₂O), ppm: 5.76 (s, 1H); 5.67 (s, 1H); 5.15 (s, ¹H); 4.96 (s, 1H); 4.84 (d, J=3.6 Hz, 0.8H, anomer); 4.40 (d, J=7.2 Hz, 0.2H, anomer); 3.20-4.00 (m, 10H); 2.20-2.45 (s, 2H); 1.95 (d, J=12 Hz, 2H); 1.48 (d, J=8 Hz, 2H); 1.12-1.30 (s, 2H); 0.95-1.10 (m, 2H,); MS: m/z: 652.36 [M+H]⁺

Complex 3:

¹H NMR (400 MHz, D₂O), ppm: 4.88 (d, J=3.6 Hz, 1H, anomer); 3.65-3.85 (m, 5H); 3.55-3.63 (m, 1H); 3.45-3.53 (m, 1H); 3.25-3.40 (m, 2H); 2.80-3.00 (m, 1H); 2.25-2.45 (m, 2H); 1.85-2.05 (m, 2H); 1.56-1.73 (m, 2H); 1.49 (d, J=8 Hz, 2H), 1.13-1.33 (m, 2H); 0.92-1.11 (m, 2H); MS: m/z: 666.65 [M+H]⁺

Complex 5:

¹H NMR (400 MHz, D₂O), ppm: 4.89 (s, 1H); 3.30-4.00 (m, 9H); 2.90-3.20 (m, 1H); 2.20-2.45 (m, 2H); 1.90-2.05 (m, 2H); 1.50 (d, J=8 Hz, 2H); 1.16-1.30 (m, 2H); 1.00-1.15 (m, 2H); MS: m/z: 652.16 [M+H]⁺

Complex 6:

¹H NMR (400 MHz, D₂O), ppm: 4.86 (s, 1H); 3.50-3.96 (m, 8H); 2.82-3.20 (m, 2H); 2.20-2.45 (m, 2H); 1.96 (d, J=12 Hz, 2H); 1.61-1.75 (m, 2H); 1.51 (d, J=6 Hz, 2H); 1.13-1.30 (m, 2H); 0.95-1.12 (m, 2H); MS: m/z: 666.18 [M+H]⁺

Complex 8:

¹H NMR (400 MHz, D₂O), ppm: 4.90 (d, J=3.6 Hz, 1H); 4.10-4.30 (m, 1H); 3.50-4.00 (m, 8H); 2.80-3.40 (m, 1H); 2.28-2.45 (m, 2H); 1.90-2.00 (m, 2H); 1.40-1.60 (m, 2H); 1.16-1.30 (br, 2H); 1.00-1.15 (m, 2H); MS: m/z: 652.33 [M+H]⁺

Complex 9:

¹H NMR (400 MHz, D₂O), ppm: 4.90 (d, J=4 Hz, 1H); 3.62-4.00 (m, 7H); 3.50-3.60 (m, 1H); 2.70-3.00 (m, 2H); 2.20-2.40 (m, 2H); 1.90-2.10 (m, 2H); 1.60-1.70 (m, 2H); 1.50 (d, J=6 Hz, 2H); 1.18-1.30 (m, 2H); 1.00-1.16 (m, 2H); MS: m/z: 666.20 [M+H]⁺

Complex 10:

¹H NMR (400 MHz, D₂O), ppm: 4.88 (d, J=3.6 Hz, 0.8H); 4.45 (d, J=7.2 Hz, 0.2H); 3.00-4.50 (m, 8H); MS: m/z: 558.13 [M+H]⁺

Complex 11:

¹H NMR (400 MHz, D₂O), ppm: 4.88 (d, J=3.6 Hz, 0.8H, anomer); 4.42 (d, J=7.2 Hz, 0.2H, anomer); 3.15-3.95 (m, 10H); MS: m/z: 572.11 [M+H]⁺

Complex 12:

¹H NMR (400 MHz, D₂O), ppm: 4.87 (d, J=3.6 Hz, 1H, anomer); 3.64-3.83 (m, 5H); 3.55-3.63 (m, 1H); 3.43-3.53 (m, 1H); 3.26-3.40 (m, 2H); 2.80-2.98 (m, 1H); 1.60-1.75 (m, 2H); MS: m/z: 586.56 [M+H]⁺

Complex 14:

¹H NMR (400 MHz, D₂O), ppm: 4.85 (s, 1H); 3.50-3.95 (m, 9H); 2.28-3.20 (m, 1H); MS: m/z: 572.21 [M+H]⁺

Complex 15:

¹H NMR (400 MHz, D₂O), ppm: 4.90 (s, 1H); 3.50-4.00 (m, 8H); 2.80-3.20 (m, 2H); 1.60-1.73 (m, 2H); MS: m/z: 586.17 [M+H]⁺

Complex 17:

¹H NMR (400 MHz, D₂O), ppm: 4.89 (d, J=3.6 Hz, 1H); 3.50-4.20 (m, 9H); 2.80-3.40 (m, 1H); MS: m/z: 572.21 [M+H]⁺

Complex 18:

¹H NMR (400 MHz, D₂O), ppm: 4.90 (d, J=4 Hz, 1H); 3.50-4.00 (m, 8H); 2.68-3.10 (m, 2H); 1.55-1.75 (m, 2H); MS: m/z: 586.19 [M+H]⁺

Complex 19:

¹H NMR (400 MHz, D₂O), ppm: 4.88 (d, J=3.6 Hz, 0.8H, anomer); 4.83 (br, 4H); 4.44 (d, J=7.2 Hz, 0.2H, anomer); 3.00-4.30 (m, 8H); 2.41 (m, 2H); 1.15-1.30 (m, 12H); MS: m/z: 642.21 [M+H]⁺

Complex 20:

¹H NMR (400 MHz, D₂O), ppm: 4.85 (d, J=4 Hz, 1H); 3.40-4.10 (m, 9H); 2.95-3.20 (m, 1H); MS: m/z: 556.28 [M+H]⁺

Complex 21:

¹H NMR (400 MHz, D₂O), ppm: 4.85 (s, 1H); 3.40-4.10 (m, 9H); 2.95-3.20 (m, 1H); MS: m/z: 556.28 [M+H]⁺

Complex 23:

¹H NMR (400 MHz, D₂O), ppm: 4.85 (s, 1H); 3.40-4.10 (m, 9H); 2.95-3.20 (m, 1H); MS: m/z: 656.21 [M+H]⁺

Complex 24:

¹H NMR (400 MHz, D₂O), ppm: 4.85 (s, 1H); 3.40-4.10 (m, 9H); 2.95-3.20 (m, 1H); MS: m/z: 670.28 [M+H]⁺

Complex 26:

¹H NMR (400 MHz, D₂O), ppm: 4.85 (s, 1H); 3.40-4.10 (m, 9H); 2.95-3.20 (m, 1H); MS: m/z: 656.23 [M+H]⁺

Complex 27:

¹H NMR (400 MHz, D₂O), ppm: 4.85 (s, 1H); 3.40-4.10 (m, 9H); 2.95-3.20 (m, 1H); MS: m/z: 670.20 [M+H]⁺

Complex 29:

¹H NMR (400 MHz, D₂O), ppm: 4.87 (d, J=3.6 Hz, 0.8H); 4.83 (br, 4H); 4.42 (d, J=7.2 Hz, 0.2H); 3.00-4.15 (m, 10H); 2.68-2.79 (m, 2H); 0.75-0.95 (m, 8H); MS: m/z: 652.31 [M+H]⁺

### EXAMPLE 5

The preparation of the representative platinum complexes The preparation of the complex 2:

(1) Preparation of 1-O-D-glucoside-2-bromoethane (IV-2):

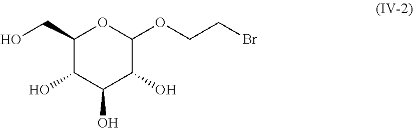 [SMILES: OC[C@H]1OC(OCCBr)[C@H](O)[C@@H](O)[C@@H]1O]

1) To 2-bromoethanol (10 mL) was added glucose (2.7 g) at room temperature, and then cooled to 0° C. The air inside the flask was replaced with nitrogen, then 1 mL of BF₃-Et₂O complex was added dropwise under a nitrogen atmosphere;

2) The reaction solution was stirred at 0° C. for 15 minutes, then slowly warmed to room temperature and stirred for 30 minutes, then heated to 80° C. and stirred for 5 hours; After completion of the reaction, the solvent was evaporated in vacuum and the residue was simply subjected to purification on silica gel chromatography (CH₂Cl₂/CH₃OH: 6/1) to give the crude product (IV-2). Yield: 2.3 g. MS, m/z: 287. 23 [M+H]⁺

(2) Preparation of 1-O-(2,3,4,6-tetra-acetyl-D-glucoside)-2-bromoethane (V-2):

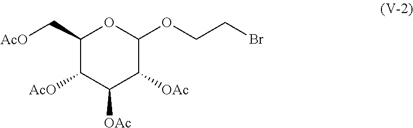 [SMILES: CC(=O)OC[C@H]1OC(OCCBr)[C@H](OC(C)=O)[C@@H](OC(C)=O)[C@@H]1OC(C)=O]

2.3 g of 1-O-D-glucoside-2-bromoethane (IV-2) obtained in the previous step was dissolved in pyridine and acetic anhydride (7 mL: 7 mL) at room temperature, then the reaction mixture was stirred overnight and the reaction was monitored by TLC. After completion of the reaction, ethyl acetate (100 mL) was added, washed with 5% (volume concentration) aqueous hydrochloric acid (2×25 mL), re-extracted with ethyl acetate (2×25 mL), then the organic phase was combined and washed with saturated NH₄Cl (aq.) (1×100 mL), water (1×100), saturated NaHCO₃ (aq.) (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as a colorless oil (V-2). Yield: 2.5 g.

¹H NMR (400 MHz, CDCl₃): δ5.45 (t, J=9.6 Hz, 1H), 5.15 (d, J=4 Hz, 1H), 5.02 (t, J=9.6 Hz, 1H), 4.80-4.83 (m, 1H), 4.19-4.23 (m, 1H), 4.04-4.15 (m, 2H), 3.92-4.00 (m, 1H), 3.75-3.85 (m, 1H), 3.49 (t, J=6 Hz, 2H), 1.91-2.11 (m, 12H). MS, m/z: 455.15 [M+H]⁺

(3) Preparation of 1-O-(2,3,4,6-tetra-acetyyl-D-glucoside)-propane-3,3-diethyl dicarboxylate (VI-2):

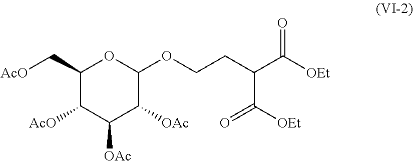 [SMILES: CCOC(=O)C(CCOC1O[C@H](COC(C)=O)[C@@H](OC(C)=O)[C@H](OC(C)=O)[C@H]1OC(C)=O)C(=O)OCC]

2.5 g of 1-O-(2,3,4,6-tetra-acetyl-D-glucoside)-2-bromoethane (V-1) obtained in the previous step was dissolved in dry DMF (5 mL), to which was added potassium carbonate (3 g) followed by diethyl malonate (1.76 g), then the reaction mixture was stirred at room temperature overnight and the reaction was monitored by TLC. After completion of the reaction, ethyl acetate (100 mL) was added, washed with saturated NH₄Cl (aq.) (1×50 mL), re-extracted with ethyl acetate (2×25 mL), then the organic phase was combined and washed with saturated NH₄Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as a colorless oil (VI-2). Yield: 2.6 g.

¹H NMR (400 MHz, CDCl₃): δ5.42 (t, J=9.6 Hz, 1H), 4.96-5.10 (m, 2H), 4.78-4.90 (m, 1H), 4.03-4.33 (m, 5H), 3.92-4.02 (m, 1H), 3.71-3.87 (m, 1H), 3.71-3.87 (m, 1H), 3.55 (t, J=8 Hz, 1H), 3.40-3.50 (m, 1H), 2.13-2.28 (m, 2H), 1.94-2.14 (m, 12H), 1.15-1.35 (m, 6H). MS, m/z: 535.34 [M+H]⁺

(4) Preparation of 1-O-(2,3,4,6-tetra-acetyl-D-glucoside)-propane-3-chloro-3,3-diethyl dicarboxylate (VII-2):

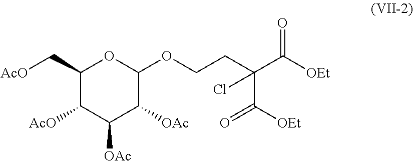 [SMILES: CCOC(=O)C(Cl)(CCOC1O[C@H](COC(C)=O)[C@@H](OC(C)=O)[C@H](OC(C)=O)[C@H]1OC(C)=O)C(=O)OCC]

2.6 g of 1-O-(2,3,4,6-tetra-acetyl-D-glucoside)-propane-3,3-diethyl dicarboxylate was dissolved in dry THF (20 mL), and then cooled to 0° C. The air inside the flask was replaced with nitrogen, and to the above solution was slowly added solid sodium hydride (235 mg, 60% suspension in mineral oil) under a nitrogen atmosphere and stirred for 1 hour after warming to room temperature, then NCS (780 mg) was added and stirred for 2 hours at room temperature, the solvent was removed by rotary evaporation. Ethyl acetate (100 mL) was added, washed with saturated NH₄Cl (aq.) (1×50 mL), re-extracted with ethyl acetate (2×25 mL), then the organic phase was combined and washed with saturated NH₄Cl (aq.) (1×100 mL), water (1×100 mL), brine (1×100 mL), dried over anhydrous sodium sulfate and concentrated under reduced pressure to give the pale yellow crude product. The obtained crude product was purified by silica gel column chromatography (PE/EtOAc: 3/1) to give the desired product as a colorless oil (VII-2). Yield: 2.6 g.

¹H NMR (400 MHz, CDCl₃): δ5.29 (t, J=9.6 Hz, 1H), 4.90-5.00 (m, 2H), 4.67-4.78 (m, 1H), 4.15-4.35 (m, 5H), 3.97-4.05 (m, 2H), 3.85-3.95 (m, 1H), 3.45-3.55 (m, 1H), 2.48-2.65 (m, 2H), 1.85-2.05 (m, 12H), 1.10-1.30 (m, 6H). MS, m/z: 569.19 [M+H]⁺

(5) Preparation of 1-O-D-glucoside-propane-3-chloro-3,3-dicarboxylic Acid (III-2):

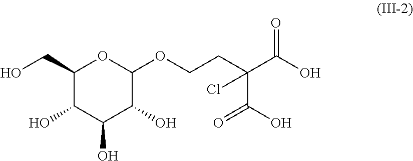 [SMILES: O=C(O)C(Cl)(CCOC1O[C@H](CO)[C@@H](O)[C@H](O)[C@H]1O)C(=O)O]

1) 2.6 g of 1-O-(2,3,4,6-tetra-acetyl-D-glucoside)-propane-3-chloro-3, 3-diethyl dicarboxylate was dissolved in methanol (5 mL), to which was then added a solution of sodium hydroxide (1.5 g) dissolved in water (10 mL) at room temperature and then heated at 60° C. for 24 hours. The reaction was monitored by TLC.

2) After completion of the reaction, the methanol was removed by rotary evaporation, then the residue was treated with strong acid cation exchange resin. The aqueous filtrate obtained from filtration of the resin was lyophilized to give a colorless viscous liquid (1.5 g). The crude product was used directly in the next step. MS, m/z: 345.11 [M+H]⁺

(6) Preparation of cis-[trans-(1R, 2R)-diaminocyclohexane] Pt (II) (1-O-D-glucoside-propane-3-chloro-3,3-dicarboxylate) (I-2):

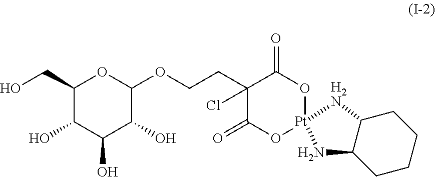

1) 1.5 g of crude 1-O-D-glucoside-propane-3-chloro-3,3-dicarboxylic acid was dissolved in water (15 mL), to which was added a freshly prepared aqueous solution of barium hydroxide to adjust the pH to 7, and then stirred at room temperature for 30 minutes.

2) Under the protection of nitrogen, to the reaction solution of 1) was added a solution of trans-(1R,2R)-diaminocyclohexane platinum sulfate (1.7 g) dissolved in water (2 mL) followed by a freshly prepared aqueous solution of barium hydroxide to re-adjust the pH to 7. The reaction mixture was stirred in the dark at a room temperature overnight.

3) After completion of the reaction, the precipitate was removed by centrifugation, the supernatant was collected and purified by semi-preparative HPLC, and the fraction of the collected product was lyophilized by a lyophilizer to obtain the final product (I-2) as a white solid. Yield: 1.5 g.

¹H NMR (400 MHz, D₂O): δ5.76 (s, 1H), 5.67 (s, 1H), 5.15 (s, 1H), 4.96 (s, 1H), 4.84 (d, J=3.6 Hz, 0.8H, α-isomer), 4.40 (d, J=7.2 Hz, 0.2H, β-isomer), 3.20-4.00 (m, 10H), 2.20-2.45 (s, 2H), 1.95 (d, J=12 Hz, 2H), 1.48 (d, J=8 Hz, 2H), 1.12-1.30 (s, 2H), 0.95-1.10 (m, 2H). MS, m/z: 652.36 [M+H]⁺

1-10. (canceled)

11. A method for treating or preventing cancer in a patient, comprising administering a therapeutical composition containing a water soluble platinum complex of formula (I): 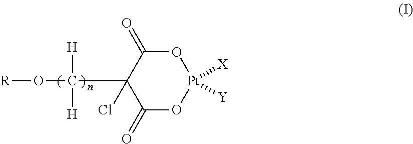
Wherein:
X and Y are ligand. X and Y are same or independently chosen from isopropylamine, cyclopropylamine, or X and Y together form a 1,2-cyclohaxanediamine chosen from trans-(1R,2R)-cyclohexanediamine, trans-(1S,2 S)-cyclohexanediamine, cis-(1R,2S)-cyclohexanediamine, cis-(1S-2R)-cyclohexanediamine, trans-meso-1,2-cyclohexanediamine, cis-meso-1,2-cyclohexanediamine;
n is an integer from 1 to 6;
R is chosen from 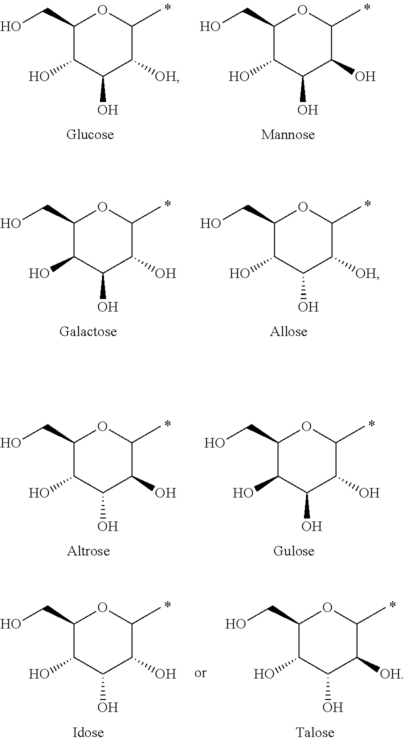
wherein the monosaccaride represents the isomer of—or—or mixture of both;
The cancer is selected from the group consisting of human non-small cell lung cancer, human prostate cancer, human breast cancer, human ovarian cancer, human leukemia cancer or human colorectal cancer.

12. The method for treating and preventing cancer with the platinum complex of claim 11, wherein R is chosen from: 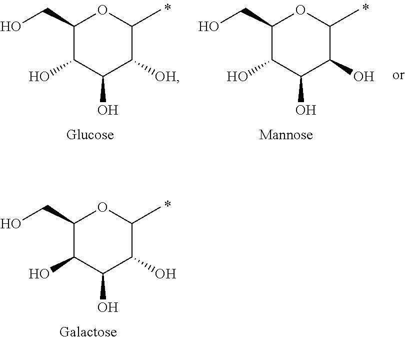
wherein the monosaccaride represents the isomer of—or—or mixture of both;

13. The method for treating and preventing cancer with the platinum complex of claim 11, wherein X and Y together form trans-(1R,2R)-cyclohexanediamine;

14. A method for treating or preventing cancer in a patient, comprising administering a therapeutical composition containing a water soluble platinum complex of formula (I): 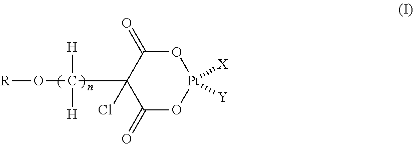
Wherein:
X and Y are ligand. X and Y are same or independently chosen from isopropylamine, cyclopropylamine, or X and Y together form a 1,2-cyclohaxanediamine chosen from trans-(1R,2R)-cyclohexanediamine, trans-(1S,2 S)-cyclohexanediamine, cis-(1R,2S)-cyclohexanediamine, cis-(1S-2R)-cyclohexanediamine, trans-meso-1,2-cyclohexanediamine, cis-meso-1,2-cyclohexanediamine;
n is an integer from 1 to 6;
R is chosen from 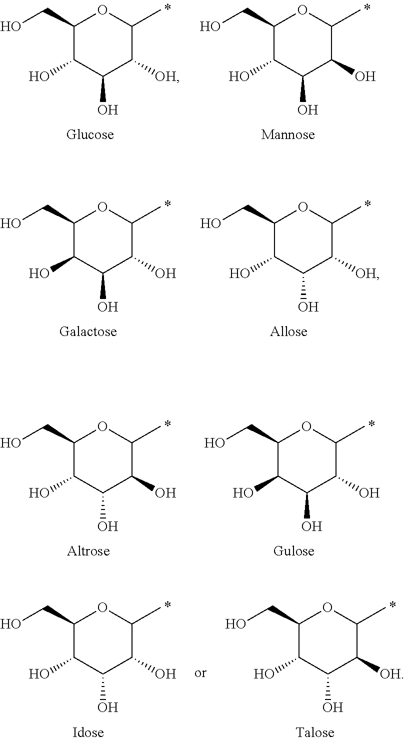
wherein the monosaccaride represents the isomer of—or—or mixture of both;
with one or more further chemotherapeutic agents chosen from the group consisting of Cisplatin, Transplatin, trans-diaminetetrachloroplatinum (IV), Carboplatin, Oxaliplatin, 5-FU, Deoxifluridine, Tegafur, Gemcitabine, Capecitabine, Clofarabine, Temozolomide, Lonafarnib, Erlotinib, Sorafenib, Sunitinib, Imatinib, Erlotinib, Bortezomib, Gimatecan, Vinblastine, Vinorelbine, Folinic Acid, Doxorubicin, Paclitaxel, Docetaxel and their diravertives, Tamoxifen, Raloxifene, Spiramycin, Irinotecan. The cancer is selected from the group consisting of human non-small cell lung cancer, human prostate cancer, human breast cancer, human ovarian cancer, human leukemia cancer or human colorectal cancer.

15. The method of claim 14, wherein X and Y together form trans-(1R, 2R)-cyclohexanediamine;

16. The method of claim 14, wherein the further chemotherapeutic agent is one or more members selected from 5-FU and Folinic Acid.
